(19) United States
(12) Patent Application Publication
(10) Pub. No.: US 2022/0348859 A1
(43) Pub. Date: Nov. 3, 2022

(54) Lithographic masking for spatially localized biochemical stimulus delivery

(71) Applicant: The Government of the United States of America, as represented by the Secretary of the Navy, Arlington, VA (US)

(72) Inventors: Keith E. Whitener, Alexandria, VA (US); Dhanya Haridas, Ashburn, VA (US); Jillian N. Lanese, Webster, NY (US); Woo-Kyung Lee, Vienna, VA (US)

(73) Assignee: The Government of the United States of America, as represented by the Secretary of the Navy, Arlington, VA (US)

(21) Appl. No.: 17/714,224

(22) Filed: Apr. 6, 2022

Related U.S. Application Data
(60) Provisional application No. 63/183,323, filed on May 3, 2021.
(60) Provisional application No. 63/309,540, filed on Feb. 12, 2022.

(51) Int. Cl. C12M 1/42; C12M 1/12; C12M 1/00

(52) U.S. Cl. CPC C12M 35/02; C12M 23/04; C12M 23/20; C12M 23/22; C12M 23/38; C12M 25/06; C12M 47/02

(57) ABSTRACT

A method of lithographic masking for spatially localized biochemical stimulus delivery, comprising the steps of providing a group of cells on a substrate, coating a layer of gelatin on a portion of the cells, creating a mask layer on a portion of the layer of gelatin on a portion of the cells, and creating an area of masked cells and an area of unmasked cells. Further, the method can include delivering a biochemical signal to the area of unmasked cells, removing the mask layer, and allowing the cells with the biochemical signal and the cells without the biochemical signal to interact freely.

### REFERENCE TO RELATED APPLICATION

This application is a non-provisional of, and claims priority to and the benefits of, U.S. Provisional Patent Application No. 63/183,323 filed on May 3, 2021, and U.S. Provisional Patent Application No. 63/309,540 filed on Feb. 12, 2022, the entireties of each are herein incorporated by reference.

### BACKGROUND

This disclosure concerns lithographic masking for spatially localized biochemical stimulus delivery.

Decoding cell-cell communication under both native and pathological conditions is of dire importance as it contributes to developing a better understanding of the various biological processes involved.

Studying cell-cell communication is becoming more imperative as we now understand that cells are heterogeneous in nature and spatial gradients of signaling molecules control the response/fate of the adjoining cells.

This observation provides an opportunity for developing synthetic molecules that can mimic these responses in vitro. However, studying single-cell communication in the native environment is challenging and limited by the tools that are available.

Currently, one of the most commonly used techniques to study different cell populations is using a microfluidics system, a miniaturized process involving small fluid volumes (microliters to picoliters) within a fabricated system. Microfluidics has gained extensive acceptance in the field of biotechnology for various applications which include single-cell functional proteomics, droplet-based monoclonal antibody screening and organ-on-a-chip platform.

However, microfluidics also present various challenges such as the need for specialized instrumentation, clogging of the microchannel with debris or bubbles and custom designing the unit to address the current needs.

Hence, there is a need to identify other methods that will enable cell-cell communication studies.

This invention described herein advances the current standard and solves long-standing problems by introducing tunably permeable membranes as cellular masks, avoiding the need for complicated microfluidic device fabrication to achieve spatially heterogeneous cell cultures.

Furthermore, this invention described herein advances the current standard and solves long-standing problems by introducing impermeable graphene-based membranes or a layer of SU-8 as cell masks that can adhere to surfaces containing biological materials such as mammalian cells via a gelatin layer. This process overcomes the challenge of developing complicated single-purpose microfluidic devices to investigate cell-cell behavior, while maintaining the ability to study populations of cells interacting with one another.

### SUMMARY OF DISCLOSURE

### Description

This disclosure concerns lithographic masking for spatially localized biochemical stimulus delivery.

This invention concerns a method to spatially isolate single cells and small groups of cells using biocompatible cell masks in combination with standard lithography methods, or graphene-based biocompatible cell masks or to use SU-8 or GelMA or similar material in combination with gelatin.

This process overcomes the challenge of developing complicated single-purpose microfluidic devices to investigate cell-cell behavior, while maintaining the ability to study populations of cells interacting with one another.

Our process was developed to build a platform that will facilitate analyzing cell-cell communication and enable cell manipulations in a temporal and spatial manner. This method also allows targeting specific populations of cells or areas of biological films that require further probing while maintaining the biological material architecture used in the process.

Our process eliminates the need to physically isolate the biological material of interest from a surface. Specific geometric patterns can be lithographed into chemically modified graphene membranes, which are then delaminated via water lift-off and physically transferred to the area(s) of interest identified on a biological substrate coated with gelatin. This creates an impermeable barrier akin to a lithography shadow mask on the cellular material.

An array of biochemically active compounds, dyes, and proteases can then be added to the cell media, exposing only those unmasked parts of the cell culture to these stimuli.

Specific geometric patterns can be lithographed into photocrosslinkable material spin-coated onto ordinary gelatin, which is then delaminated via hot water dissolution of gelatin and physically transferred to the area(s) of interest identified on a biological substrate. This creates a tunably permeable barrier akin to a lithography shadow mask on the cellular material. An array of biochemically active compounds, dyes, and proteases can then be added to the cell media, exposing the masked and unmasked parts of the cell culture to different levels of stimuli.

Alternatively, the biochemically active compounds, etc., can be packaged into the transferrable membrane to deliver the stimuli only to the masked parts of the cell culture. This process thus enables studying different cell populations or cells derived from different sources to be either placed or trypsinized adjacent to the existing cell population, thus facilitating co-culture studies, depending upon which cell population has the mask.

This binding is reversible thus providing both temporal and spatial control of the process.

This invention enables spatially heterogenous delivery of biomolecules and small molecules to single cells or populations of cells by masking them using graphene-based impermeable membranes or SU-8 or GelMA or similar material in combination with gelatin.

Here, we manipulate the local environment of the cells and pave way for cell-specific payload delivery mechanisms to investigate cell-cell communication and facilitate cell-based discoveries.

This invention has been demonstrated by localizing gelatin photo-crosslinking chemistry using graphene based masks, as well as by trypsinizing unmasked cells toward easily configurable cell co-cultures, or using photopatterned SU-8 or GelMA as diffusion barriers.

### DESCRIPTION OF THE DRAWINGS

The following description and drawings set forth certain illustrative implementations of the disclosure in detail, which are indicative of several exemplary ways in which the various principles of the disclosure may be carried out. The illustrated examples, however, are not exhaustive of the many possible embodiments of the disclosure. Other objects, advantages and novel features of the disclosure will be set forth in the following detailed description when considered in conjunction with the drawings.

FIG. 1 illustrates masking of cells: delivery of biochemical payload to unmasked cells. Chemically modified graphene or mask/gelatin layer on a portion of a cell culture masks the cells under the membrane. Chemical or biological information is delivered to unmasked cells, while the graphene or mask/gelatin membrane protects the masked cells from payload delivery. Gelatin layer melts off at 37° C., yielding a spatially heterogeneous cell culture.

FIG. 2 illustrates masking of cells with chemically modified graphene: delivery of biochemical payload to masked cells. Chemically modified graphene with a gelatin layer on a portion of a cell culture. In this variation, the gelatin or the chemically modified graphene carries the biochemical payload to be delivered to the cells. After incubation at 37° C., the gelatin melts off and the masked cells now contain new biochemical information, yielding a spatially heterogeneous cell culture.

FIG. 3 illustrates graphene masks for fabrication of cell co-cultures. Masking of cells with chemically modified graphene and gelatin. Trypsin incubation or laser ablation to remove cells from unmasked area. Introduction of second cell population to masked cell culture. Removal of mask at 37° C. by melting gelatin to produce spatially defined cell co-culture.

FIG. 4 illustrates examples of reduction to practice of graphene masks. Other examples of reduction to practice of masks include SU-8 photoresist on gelatin. Masked exposure of cells to small dye molecule. Laser ablation-based mask-facilitated co-culture of fibroblasts and mesenchymal stem cells. Trypsin-based mask-facilitated cell liftoff. Transfer of photoresist to curved surface. Evaporation of gold through SU-8 windows.

### DETAILED DESCRIPTION OF THE INVENTION

This disclosure teaches methods and devices for lithographic masking for spatially localized biochemical stimulus delivery.

This invention concerns a method to spatially isolate single cells and small groups of cells using biocompatible cell masks in combination with standard lithography methods, or graphene-based biocompatible cell masks or to use SU-8 or GelMA or similar material in combination with gelatin.

This process overcomes the challenge of developing complicated single-purpose microfluidic devices to investigate cell-cell behavior, while maintaining the ability to study populations of cells interacting with one another.

Our process was developed to build a platform that will facilitate analyzing cell-cell communication and enable cell manipulations in a temporal and spatial manner. This method also allows targeting specific populations of cells or areas of biological films that require further probing while maintaining the biological material architecture used in the process.

Our process eliminates the need to physically isolate the biological material of interest from a surface. Specific geometric patterns can be lithographed into chemically modified graphene membranes, which are then delaminated via water lift-off and physically transferred to the area(s) of interest identified on a biological substrate coated with gelatin. This creates an impermeable barrier akin to a lithography shadow mask on the cellular material.

An array of biochemically active compounds, dyes, and proteases can then be added to the cell media, exposing only those unmasked parts of the cell culture to these stimuli.

Specific geometric patterns can be lithographed into photocrosslinkable material spin-coated onto ordinary gelatin, which is then delaminated via hot water dissolution of gelatin and physically transferred to the area(s) of interest identified on a biological substrate. This creates a tunably permeable barrier akin to a lithography shadow mask on the cellular material. An array of biochemically active compounds, dyes, and proteases can then be added to the cell media, exposing the masked and unmasked parts of the cell culture to different levels of stimuli.

Alternatively, the biochemically active compounds, etc., can be packaged into the lithographed membrane to deliver the stimuli only to the masked parts of the cell culture. This process thus enables studying different cell populations, cells derived from different sources to be either placed or trypsinized adjacent to the existing cell population, thus facilitating co-culture studies, depending upon which cell population has the mask.

This binding is reversible and time dependent thus providing both temporal and spatial control of the process.

This invention enables spatially heterogenous delivery of biomolecules and small molecules to single cells or populations of cells by masking them using graphene-based impermeable membranes or SU-8 or GelMA or similar material in combination with gelatin.

Here, we manipulate the local environment of the cells and pave way for cell-specific payload delivery mechanisms to investigate cell-cell communication and facilitate cell-based discoveries.

This invention has been demonstrated by localizing gelatin photo-crosslinking chemistry using graphene-based masks, as well as by trypsinizing unmasked cells toward easily configurable cell co-cultures, or using SU-8 or GelMA.

### Example 1

Reduced graphene oxide membranes are highly impermeable to a wide variety of biologically relevant molecules.

Graphene can be lithographed into arbitrary patterns using standard methods and transferred onto arbitrary substrates using water delamination.

Gelatin is coated on top of the cell surface to serve as an adherent layer for graphene as well as maintain a viable environment for the cells.

Prepatterned graphene oxide membrane is transferred on to a preselected area on the gelatin layer to serve as a mask for the cells in that area.

Further manipulations can be performed on the masked or unmasked cells. This process enables isolation and investigation of a chosen population of cells, among different cell populations on a single platform.

The invention described herein advances the current standard by introducing the use of impermeable graphene-based membranes as cellular masks, avoiding the need for complicated microfluidic device fabrication to achieve spatially heterogeneous cell cultures.

### Example 2

Our process employs our previously-described water lift-off transfer of chemically modified graphene from a preparation substrate onto an arbitrary target substrate. In this case, biological materials (e.g., mammalian cells, bacterial biofilms, single layer tissue or organ slices) prepared on an appropriate surface (e.g., glass coverslips or petri dishes) are the target substrate, and a thin coat of 15% (w/v) gelatin is added either on top of the cell culture or on top of the graphene surface to ensure both adhesion of the graphene-based membrane as well as to enhance biocompatibility of the graphene with the cells.

### Example 3

The invention consists of the use of impermeable graphene-based membranes as masks to cover specific groups of cells growing on a surface. This process thus creates 2 regions, a region of cells that are masked by graphene and another that is not. The graphene-based membranes are prepared using standard lithographic methods to have a specific shape: an overall geometric configuration, open windows in the membrane, or both. The material used in the graphene membranes consists either of hydrogenated graphene or thermally- or chemically-reduced graphene oxide.

Typical procedures for each of these transfer layer preparations are as follows.

### Example 4

### For Hydrogenated Graphene

Single layer graphene was grown via chemical vapor deposition and transferred onto silicon/silicon oxide wafers using a standard polymer-supported copper etching process. The graphene was then hydrogenated via the Birch reduction, a dissolving metal hydrogenation. This reaction has been shown to weaken the adhesion of graphene to its substrate and allow the transfer of chemical functionality from one substrate to another.

### Example 5

### For Graphene Oxide

Graphene oxide was prepared from graphite via the Hummers method. A suspension of 8 g/L of graphite oxide in water was diluted by 50% (v/v) with methanol and spin-coated at 900-3000 rpm or drop-cast onto a hydrophilic plasma-cleaned glass surface.

The graphene oxide was then either thermally or chemically reduced. Thermal reduction consisted of heating the graphene oxide to 250° C. in air for 30 seconds. Chemical reduction consisted of exposing the graphene oxide to hydrazine vapor in a small chamber for 15 minutes.

### Typical Lithography Procedures are as Follows.

### Example 6

A photocurable compound such as SU-8 is spin-coated onto the graphene-based membrane with a thickness of 1.5 μm. SU-8 is an epoxy-based negative photoresist which cross-links when exposed to UV light. The photolithography is performed with a mask aligner and the sample is exposed to UV light through a shadow mask for 15 seconds.

After a post-bake at 95° C. for 90 seconds, the patterns are then developed in SU-8 developer solution, followed by rinsing with isopropyl alcohol. This process removes the regions of SU-8 unexposed to UV light, while the graphene membrane underneath remains intact.

A typical transfer involves immersing the lithographed graphene-based membrane in distilled water or phosphate-buffered saline to delaminate it from its substrate. The membrane is then relaminated onto the target substrate, a bed of cells with a thin coat of gelatin, and placed into cell growth media.

This process thus ensures that only the selected area of cells (masked or without mask) are subjected to manipulation.

Cellular manipulations can then be performed which include co-culture experiments or delivering molecules of interest to either the masked or unmasked cells.

If desired, after cell manipulation is complete, the system can be warmed to standard incubator temperatures of 37° C., at which point the gelatin melts off, removing the graphene-based mask.

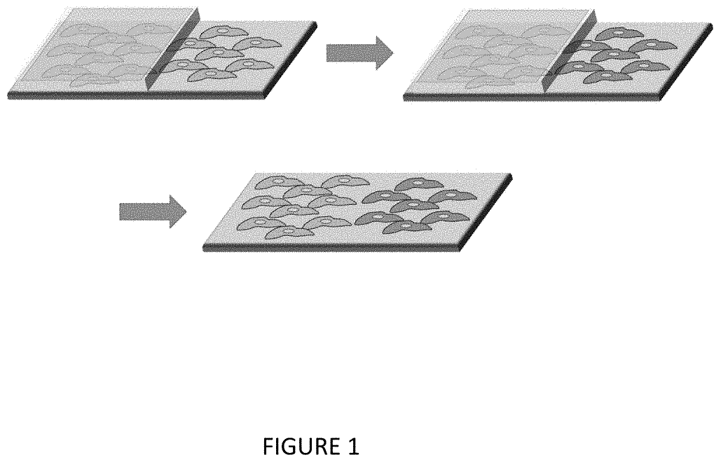

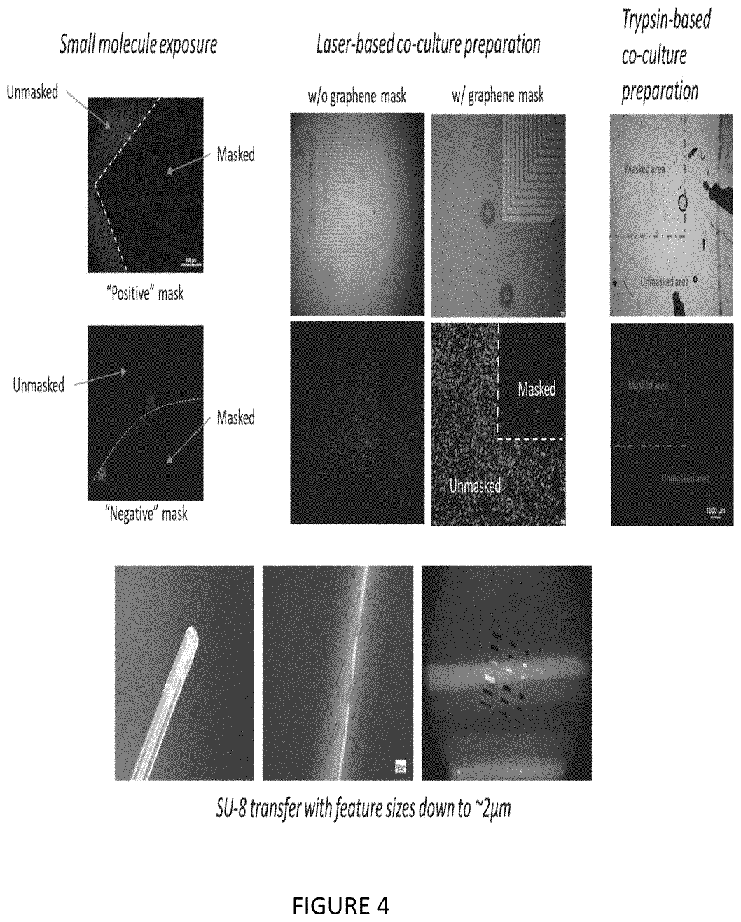

A schematic of the process for using graphene masks to deliver biochemical information to unmasked cells is given in FIG. 1, while an example of using the masks to deliver information to the masked cells is given in FIG. 4.

In both cases, the spatial heterogeneity induced by the graphene masks is the key to encoding spatially localized information in the cell population.

### Example 7

This invention consists of the use of membranes as masks to cover specific groups of cells growing on a surface.

The mask material can include standard negative or positive photoresists (e.g., SU-8, Shipley, etc.) and photocrosslinkable gelatin (GelMA).

This process thus creates 2 regions, a region of cells that are masked and another that is not. The membranes are prepared using standard photolithography into a specific shape, potentially including an overall geometric configuration, open windows in the membrane, or both.

### Example 8

Typical lithography procedures with SU-8 as an example are as follows.

A photocurable compound such as SU-8 is spin-coated directly onto gelatin with a thickness of 1.5 μm. SU-8 is an epoxy-based negative photoresist which cross-links when exposed to UV light. The photolithography is performed with a mask aligner and the sample is exposed to UV light through a shadow mask for 15 seconds.

After a post-bake at 95° C. for 90 seconds, the patterns are then developed and rinsed with isopropyl alcohol.

This process removes the regions of SU-8 unexposed to UV light.

A typical transfer involves immersing the lithographed membrane in hot (>60° C.) water to delaminate it from its substrate.

The membrane is then relaminated onto the target substrate, which can include a bed of cells with a thin coat of gelatin, and placed into cell growth media. This process thus ensures that only the selected area of cells (masked or without mask) are subjected to manipulation.

In place of impermeable masks such as SU-8, one can use a permeable semi-permanent cell-compatible material such as photocrosslinkable gelatin methacrylate (GelMA) as a mask.

This material can be pre-loaded with biochemicals to enable stimuli to be delivered by chemical leaching out of the mask.

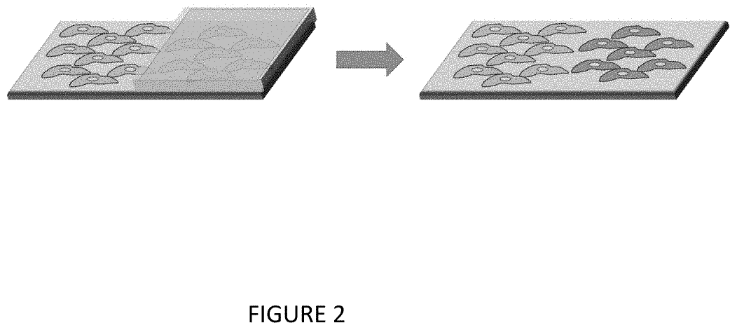

A schematic of the process for using masks to deliver biochemical information to masked cells is given in FIG. 2, while examples of reduction to practice (without cell integration) are given in FIG. 4. In both cases, the spatial heterogeneity induced by the masks is the key to encoding spatially localized information in the cell population.

### Example 9

We can achieve trypsin-mediated cell co-cultures using this process as well. The culture with the graphene mask is treated with trypsin, which lyses the adhesion of cells to their substrates and allows them to lift off the surface of the target substrate. Since the trypsin cannot penetrate the mask, only the unmasked cells are lifted off. These cells are then discarded and a different population of cells is seeded. Those cells seed everywhere, including on top of the mask. When the temperature is raised to remove the graphene mask, one is left with a heterogeneous cell co-culture. An example of reduction to practice of spatially selective cell liftoff is given in FIG. 4.

### Example 10

We can further achieve micron scale resolution of cell masking by using the NRL-patented technology BioLP (Biological Laser Printing), which has the ability to excise membranes (i.e chemically modified graphene) with high spatial resolution using principles similar to that of laser induced forward transfer. The BioLP system has currently been reconstructed with improved motion controls for higher fidelity, a user friendly computer interface and higher resolution video capture.

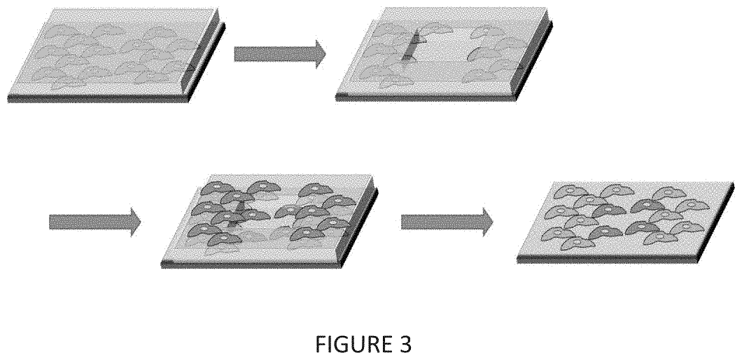

A schematic of the use of this process for producing cell co-cultures is given in FIG. 3, while examples of reduction to practice are given in FIG. 4.

### Example 11

A process tethering a thin (<100 nm) film of impermeable chemically modified graphene-derived material (e.g., hydrogenated graphene, reduced graphene oxide, etc.) to a culture of living cells on a substrate via a thin (<10 μm) layer of gelatin to enhance cell viability.

To explore cell-interaction studies, co-culture systems such as microfluidics and transwell systems have been extensively used. There are, however, limitations to both of the commonly used systems. One of the major challenges is the lack of uniformity or consistency in a) experimental protocols and b) data acquisition methods used across different microfluidic chips or transwell systems. The other significant drawback while using a microfluidic system is the inability to scale up volumes or cell numbers. Co-culture systems are dependent on the presence of a physical barrier that keeps the cells confined in their spaces. In the proposed process, we can identify and specifically select which cells we would like to limit or manipulate from the study without the need of predesigning a physical membrane. This further enables us to scale up the system by several fold. This process also enables temporal control as mask binding is a reversible process and the cells can be unmasked whenever desired. Hence, the process provides significant advantages over the existing co-culture systems, and solves long-standing problems.

This invention has been demonstrated using mammalian cells. Specific areas coated with cells have been successfully masked with reduced graphene oxide membranes and cells in the unmasked area have been manipulated without disturbing the masked cells. Demonstrated manipulations include enzyme digestion and detachment of sub-populations of cells from their substrate, delivery of dye molecules to specific cell populations as a proof of principle, and spatially directed photo-crosslinking of gelatin for creating more permanent biomaterials integrated with a cell population.

This impermeable membrane can be used to selectively inhibit delivery of small molecules and biomolecules to cells directly under the membrane.

The impermeable membrane can be used to selectively block exposure of cells under the membrane to trypsin or other proteases, inhibiting their ability to detach from the underlying substrate while selectively detaching unmasked cells.

This invention also teaches the culturing of a second population of cells on a masked, trypsinized sample, and the subsequent mild thermal liftoff of the gelatin/graphene with the concomitant removal of the cells on top of the gelatin/graphene, thus forming a spatially patterned co-culture, which solves long-standing problems in the art.

Some additional advantages of the method and product described herein are as follows.

This process and product provides the flexibility to analyze a single cell or a population of cells without perturbing the cell/microbial layer globally.

This invention facilitates co-culture studies by enabling the removal of the unmasked cells and introducing multiple populations of cells in regions adjacent to the existing masked cells to study cell-cell communication.

This process does not require the design and synthesis of bespoke microfluidics devices, thus eliminating additional manufacturing and sterilizing costs.

This process allows for and can be used with an additional facile deposition and etching lithography technique on curved surfaces, both biological and abiological in nature.

This process can be proportionally scaled up or down, long-standing problems for existing methods, as current designs are generally limited to smaller volumes and limited spaces.

This process and product enables perfusion and signal gradient studies by incorporating sensors/biomolecule packages into the graphene layer, which is a limitation when using microfluidic chips as it dramatically increases the complexity of microfluidic device design and operation.

This invention concerns unparalleled control over cell-cell interactions to guide tissue formation and healing, thus paving way for new therapies in regenerative medicine.

This invention promotes fabrication of cost-effective biocompatible cell masks to probe and gain insight into novel biochemical signaling mechanisms.

This invention teaches design and development of unique cell differentiation patterns to further evaluate cell signaling.

Commercial applications include but are not limited to development of platforms for investigating single cell communication and evaluating cell response upon exposure to a wide array of biomolecules/chemicals.

The above examples are merely illustrative of several possible embodiments of various aspects of the present disclosure, wherein equivalent alterations and/or modifications will occur to others skilled in the art upon reading and understanding this specification and the annexed drawings. In addition, although a particular feature of the disclosure may have been illustrated and/or described with respect to only one of several implementations, such feature may be combined with one or more other features of the other implementations as may be desired and advantageous for any given or particular application. Also, to the extent that the terms “including”, “includes”, “having”, “has”, “with”, or variants thereof are used in the detailed description and/or in the claims, such terms are intended to be inclusive in a manner similar to the term “comprising”.

What we claim is:

1. A method of lithographic masking for spatially localized biochemical stimulus delivery, comprising the steps of:
providing a group of cells on a substrate;
coating a layer of gelatin on a portion of the cells;
creating a mask layer on a portion of the layer of gelatin on a portion of the cells; and
creating an area of masked cells and an area of unmasked cells.

2. The method of lithographic masking for spatially localized biochemical stimulus delivery of claim 1, further comprising the steps of:
delivering a biochemical signal to the area of unmasked cells;
removing the mask layer; and
allowing the cells with the biochemical signal and the cells without the biochemical signal to interact freely.

3. The method of lithographic masking for spatially localized biochemical stimulus delivery of claim 2,
wherein the mask layer comprises a layer of graphene.

4. The method of lithographic masking for spatially localized biochemical stimulus delivery of claim 2,
wherein the mask layer comprises a layer of SU-8 or wherein the mask layer comprises photocrosslinkable gelatin such as GelMA.

5. The method of lithographic masking for spatially localized biochemical stimulus delivery of claim 2,
wherein the mask layer is less than 100 nm in thickness and is impermeable.

6. The method of lithographic masking for spatially localized biochemical stimulus delivery of claim 5,
wherein the group of cells comprises living cells; and
wherein the layer of gelatin is less than 10 μm in thickness.

7. The method of lithographic masking for spatially localized biochemical stimulus delivery of claim 2, further comprising the steps of:
utilizing the mask layer to selectively inhibit delivery of molecules or biomolecules to cells under the mask layer.

8. The method of lithographic masking for spatially localized biochemical stimulus delivery of claim 2, further comprising the steps of:
utilizing the mask layer to selectively block exposure of cells under the mask layer to trypsin or other proteases;
inhibiting the cells under the mask layer from detaching from the substrate; and
detaching selectively the unmasked cells.

9. A method of lithographic masking for spatially localized biochemical stimulus delivery, comprising the steps of:
creating a first population of cells on a substrate;
coating a layer of gelatin on a portion of the first population of cells on the substrate;
creating a mask layer on a portion of the layer of gelatin on the first population of cells;
creating an area of masked cells and an area of unmasked cells;
removing or trypsinizing cells from the unmasked area;
seeding with a second population of cells;
creating a second population of cells;
removing the mask layer;
creating a spatially patterned co-culture; and
allowing cells from the first population of cells and cells from the second population of cells to interact freely.

10. The method of lithographic masking for spatially localized biochemical stimulus delivery of claim 9,
wherein the mask layer comprises a layer of graphene.

11. The method of lithographic masking for spatially localized biochemical stimulus delivery of claim 9,
wherein the mask layer comprises a layer of SU8 or wherein the mask layer comprises photocrosslinkable gelatin such as GelMA.

12. The method of lithographic masking for spatially localized biochemical stimulus delivery of claim 9,
wherein the mask layer is less than 100 nm in thickness and is impermeable.

13. The method of lithographic masking for spatially localized biochemical stimulus delivery of claim 12,
wherein the first population of cells or the second population of cells comprise living cells;
wherein the step of removing cells comprises laser ablation; and
wherein the layer of gelatin is less than 10 μm in thickness.

14. A product of the process of lithographic masking for spatially localized biochemical stimulus delivery, comprising the steps of:
creating a first population of cells on a substrate;
coating a layer of gelatin on a portion of the first population of cells on the substrate;
creating a mask layer on a portion of the layer of gelatin on the first population of cells;
creating an area of masked cells and an area of unmasked cells;
removing or trypsinizing cells from the unmasked area;
seeding with a second population of cells;
creating a second population of cells;
removing the mask layer;
creating a spatially patterned co-culture; and
allowing cells from the first population of cells and cells from the second population of cells to interact freely.
